(19) United States
(12) Patent Application Publication
(10) Pub. No.: US 2015/0315936 A1
(43) Pub. Date: Nov. 5, 2015

(54) INTEGRATED SYSTEM AND METHOD FOR THE FLEXIBLE USE OF ELECTRICITY

(71) Applicants: Georg MARKOWZ, (US); Jurgen Erwin LANG, (US); Rudiger SCHUTTE, (US); EVONIK DEGUSSA GMBH, Essen (DE)

(72) Inventors: Georg Markowz, Alzenau (DE); Jürgen Erwin Lang, Karlsruhe (DE); Rüdiger Schütte, Alzenau-Hörstein (DE)

(21) Appl. No.: 14/648,036

(22) PCT Filed: Nov. 8, 2013
(86) PCT No.: PCT/EP2013/073336

§ 371 Date: May 28, 2015

(30) Foreign Application Priority Data
Dec. 6, 2012 (DE) 10 2012 023 833.9

(51) Int. Cl. F01K 23/18; C07C 2/80; F01K 23/06; B01J 19/08; F02C 3/20; F01K 15/00

(52) U.S. Cl. CPC F01K 23/18; F02C 3/20; F01K 15/00; F01K 23/06; B01J 19/088; C07C 2/80; B01J 2219/0894; B01J 2219/0805

(57) ABSTRACT

The present invention relates to an integrated plant which comprises a plant for the electrothermic production of ethyne and a plant for electricity generation, the plant for the electrothermic production of ethyne being connected to the plant for electricity generation via a conduit and electricity being generated in the plant for electricity generation from a product gas obtained in the plant for the electrothermic production of ethyne. This integrated plant affords flexible use of electricity by a method in which, at times of a high electricity supply, the plant for the electrothermic production of ethyne is operated and at least some of the hydrogen and/or gaseous hydrocarbons obtained in addition to ethyne is stored and, at times of a low electricity supply, stored hydrogen and/or gaseous hydrocarbons are fed to the plant for electricity generation.

The present invention relates to an integrated plant and a method for the flexible use of electricity.

The use of renewable energy sources, such as wind power, solar energy and hydropower, is gaining ever-increasing significance for the generation of electricity. Electrical energy is typically supplied to a multitude of consumers over long-ranging, supra-regional and transnationally coupled electricity supply networks, referred to as electricity networks for short. Since electrical energy cannot be stored to a significant extent in the electricity network itself or not without further devices, the electrical power fed into the electricity network must be made to match the consumer-side power demand, known as the load. As is known, the load fluctuates time-dependently, in particular according to the time of day, the day of the week and also the time of year. Classically, the load variation is divided into the three ranges, base load, medium load and peak load, and electrical energy generators are, according to type, suitably used in these three load ranges. For a stable and reliable electricity supply, a continuous balance of electricity generation and electricity consumption is necessary. Possibly occurring short-term deviations are balanced out by what is known as positive or negative control energy or control power. In the case of regenerative electricity generating devices, the difficulty arises that, in the case of certain types, such as wind power and solar energy, the energy generating capacity is not available at all times and cannot be controlled in a specific way, but is for example subject to time-of-day and weather-dependent fluctuations, which are only under some circumstances predictable and generally do not coincide with the energy demand at the particular time.

The difference between the generating capacity of fluctuating renewable energy sources and the consumption at a given time usually has to be covered by other power generating plants, such as for example gas, coal and nuclear power plants. With fluctuating renewable energy sources being increasingly extended and covering an increasing share of the electricity supply, ever greater fluctuations between their output and the consumption at the particular time must be balanced out. Thus, even today, not only gas power plants but increasingly also bituminous coal power plants are being operated at part load or shut down in order to balance out the fluctuations. Since this variable operation of the power generating plants involves considerable additional costs, for some time the development of alternative measures has been investigated. As an alternative or in addition to varying the output of a power generating plant, one approach is to adapt the power required by one or more consumers (for example demand-side management, smart grids). Another approach is to store some of the power output when there are high generating outputs from renewable energy sources and retrieve it at times of low generating outputs or high consumption. For this purpose, even today pumped storage power plants are being used for example. Also under development are concepts for storing electricity in the form of hydrogen by electrolytic splitting of water.

The measures described here altogether involve considerable additional costs and efficiency-related energy losses. Against this background, there are increasing attempts to find better possibilities of balancing out the differences between electricity provision and electricity consumption that occur due to the use of renewable energy sources, in particular wind power and solar energy.

It is known from plants for producing ethyne in arc reactors that they can be adapted very well to a highly fluctuating supply of electrical energy by arcs being switched on or off. However, here there is the problem that in this case these plants have only a relatively low level of utilization, so that the investment costs in relation to the average annual amount of ethyne produced by the plant are very high if the plant is only operated when there is a surplus of electrical energy.

An estimated operating time of at most 20%, based on the maximum possible continuous use, results in unacceptably long payback times, so that these plants can only be made profitable by state intervention or applying unusual business models. This estimate is based on the assumption that the plant is only operated at times when there is a surplus from renewable energy sources.

Furthermore, it should be stated that, for the case where there is a low supply of renewable energy over a relatively long time, power generating plants must be provided that can ensure that a basic demand is covered. The provision of power plant capacities that is necessary for this must be economically viable as a business proposition or possibly funded by state provisions, since in this case too there are on the one hand comparatively high fixed costs and on the other hand a relatively low operating time.

Conventional power generating plants, i.e. power plants that are based on fossil or biogenous energy carriers or nuclear energy, can provide electrical energy on a planned basis over a long time. However, for political reasons, in particular reasons of sustainability and environmental protection, the use of plants based on fossil energy carriers or nuclear power is increasingly to be reduced in favor of electricity generators that are based on renewable energy sources. However, these electricity generators must be installed in relation to demand and for their part be able to be operated economically. As from a certain degree of installed capacity on the basis of renewable energy sources, it is economically more advisable to install storage capacity instead of further increasing renewable energy capacities, so that, at times when there is an excess of electricity from renewable energy, it can be appropriately used and stored and, at times when there is a shortfall of electricity, electricity can be provided from energy stores or conventional power generating plants. If energy consumption is expediently made more flexible, it can be assumed here that the times when there is a noticeable surplus or shortfall of electricity will become less. For these short times there is in spite of everything the necessity to safeguard the electricity supply, while accomplishing this as economically as possible.

In view of the prior art, it is thus an object of the present invention to provide an improved plant that is not affected by the disadvantages of conventional methods.

In particular, it was an object of the present invention to find ways of making it possible to increase the flexibility with regard to the storage and use of electrical energy in comparison with the prior art.

Furthermore, the plant should allow for flexible operation, so that it is possible to respond particularly flexibly to any change in the electricity supply and/or demand, in order for example to achieve economic advantages. At the same time, it should be possible for the plant to be used for storing or providing electrical energy even over relatively long periods of a high or low electricity supply.

Furthermore, the security of supply should be improved by the present invention.

The plant and the method should also have the highest possible efficiency. Furthermore, the method according to the invention should allow itself to be carried out using infrastructure that is conventional and widely available.

In addition, the method should allow itself to be carried out with the fewest possible method steps, but they should be simple and reproducible.

Further objects that are not explicitly mentioned arise from the overall context of the following description and the claims.

These and further objects that are not expressly mentioned and arise from the circumstances discussed at the beginning are achieved by an integrated plant, which integrates a plant for the electrothermic production of ethyne and a plant for electricity generation by connecting the plants via a conduit, so that a product gas that is obtained in the plant for the electrothermic production of ethyne can be used in the plant for electricity generation for the generation of electricity.

The subject matter of the present invention is accordingly an integrated plant which comprises a plant for the electrothermic production of ethyne and a plant for electricity generation and is characterized in that the plant for the electrothermic production of ethyne is connected to the plant for electricity generation via a conduit and the conduit feeds a product gas obtained in the plant for the electrothermic production of ethyne to the plant for electricity generation.

The subject matter of the present invention is also a method for the flexible use of electricity in which, in an integrated plant according to the invention, at times of a high electricity supply, the plant for the electrothermic production of ethyne is operated and at least some of the hydrogen and/or gaseous hydrocarbons obtained in addition to ethyne is stored and, at times of a low electricity supply, stored hydrogen and/or gaseous hydrocarbons are fed to the plant for electricity generation.

The integrated plant according to the invention and the method according to the invention have a particularly good range of properties, while the disadvantages of conventional methods and plants can be reduced significantly.

In particular, it has been found in a surprising way that it is thereby possible to operate a plant for the electrothermic production of ethyne with a high degree of utilization, while renewable energy sources can be economically used when there is a surplus. Furthermore, the plant allows a surplus of electricity from renewable energy sources, including wind power or photovoltaics, to be converted into a storable form.

Furthermore, electrical energy can also be provided in a particularly low-cost way when there is a relatively long period of a low supply of renewable energy.

A plant for the electrothermic production of ethyne can be operated well dynamically, and can therefore be adapted variably to the electricity supply. At the same time, the integrated plant can be used for storing or providing electrical energy even over relatively long periods of a high or low electricity supply. At the same time, surprisingly long runtimes of all the components of the integrated plant can be achieved, so that their operation can be made very economical.

It may also be provided that the plant for the electrothermic production of ethyne is of a controllable design, the control being performed in dependence on the electricity supply.

In a preferred embodiment of the method according to the invention, electricity from renewable energy sources is used for the electrothermic production of ethyne.

In addition, the method can be carried out with relatively few method steps, these being simple and reproducible.

The use of electricity from renewable energy sources enables the present integrated plant to provide chemical derivatives with little release of carbon dioxide, since the ethyne obtained can be converted into many chemically important derivatives with very high conversion rates and, in comparison with alternative starting materials, with less further energy being supplied or greater release of heat.

The integrated plant according to the invention serves for the expedient and flexible use of electrical energy, also synonymously referred to herein as electricity. The integrated plant can store electrical energy when there is a high electricity supply and feed electrical energy into an electricity network in particular when there is a low electricity supply. The term storage refers here to the capability of the plant to transform electricity into a storable form, in the present case chemical energy, when there is a high supply of electricity, while this chemical energy can be converted into electrical energy when there is a low supply of electricity. The storage may in this case take place in the form of the co-product hydrogen, which inevitably occurs in the electrothermic production of ethyne from methane or higher hydrocarbons. The storage may also take place in the form of products that are obtained in the electrothermic production of ethyne, in an endothermic conversion taking place in parallel with the formation of ethyne, for example by a conversion of two molecules of methane to ethane and hydrogen. It should be noted in this connection that two moles of methane (CH₄) have a lower energy content than for example one mole of ethane (C₂H₆) and one mole of hydrogen, so that energy can be stored by a conversion of methane into hydrogen and a hydrocarbon with two or more carbon atoms.

In conventional plants for the production of ethyne, a relatively great amount of energy is expended on processing the secondary product gases occurring, in order to optionally sell them in their pure form. In the present plant, this purification can be made very much easier by using the byproduct gases for their energy.

The integrated plant according to the invention comprises a plant for the electrothermic production of ethyne. The term electrothermic refers in this case to a method in which ethyne is produced in an endothermic reaction from hydrocarbons or coal and the heat required for carrying out the reaction is produced by electrical power. Preferably, gaseous or vaporized hydrocarbons are used, with particular preference aliphatic hydrocarbons. Particularly suitable are methane, ethane, propane and butane, in particular methane. In the electrothermic production of ethyne from aliphatic hydrocarbons, hydrogen is obtained as a co-product.

Suitable plants for the electrothermic production of ethyne are known from the prior art, for example from Ullmann's Encyclopaedia of Industrial Chemistry, Volume 1, 2012 Wiley-VCH Verlag GmbH & Co. KGaA, Weinheim, DOI: 10.1002/14356007.a01_{—}097.pub4, pages 296 to 303, from DE 1 900 644 A1 and from EP 0 133 982 A2.

The plant for the electrothermic production of ethyne preferably comprises an arc reactor. The electrothermic production of ethyne may in this case be performed in a one-stage process, in which at least one hydrocarbon is passed through the arc with a stream of gas. Alternatively, the electrothermic production of ethyne may be performed in a two-stage process, in which hydrogen is passed through the arc and, downstream of the arc, at least one hydrocarbon is fed into the hydrogen plasma produced in the arc.

The arc reactor is preferably operated with an energy density of 0.5 to 10 kWh/Nm³, particularly 1 to 5 kWh/Nm³ and in particular 2 to 3.5 kWh/Nm³, the energy density relating to the volume of gas that is passed through the arc.

The temperature in the reaction zone of the arc reactor varies on the basis of the gas flow, it being possible for up to 20,000° C. to be reached in the center of the arc and the temperature to be about 600° C. at the periphery. At the end of the arc, the average temperature of the gas is preferably in the range from 1300 to 3000° C., with particular preference in the range from 1500 to 2600° C.

The residence time of the feedstock in the reaction zone of the arc reactor is preferably in the range from 0.01 ms to 20 ms, with particular preference in the range from 0.1 ms to 10 ms and with special preference in the range from 1 to 5 ms. After that, the gas mixture emerging from the reaction zone is quenched, i.e. subjected to very rapid cooling to temperatures of less than 250° C., in order to avoid decomposition of the thermodynamically unstable intermediate product acetylene. A direct quenching process, such as for example the feeding in of hydrocarbons and/or water, or an indirect quenching process, such as for example rapid cooling in a heat exchanger with steam generation, may be used for the quenching. Direct quenching and indirect quenching may also be combined with each other.

In a first embodiment, the gas mixture emerging from the reaction zone is only quenched with water. This embodiment features relatively low investment costs. However, it is disadvantageous that in this way a considerable part of the energy contained in the product gas is not used, or is used only with a low exergetic value.

In a preferred embodiment, the gas mixture emerging from the reaction zone is mixed with a hydrocarbon-containing gas or a hydrocarbon-containing liquid, at least some of the hydrocarbons being cracked endothermically. Depending on how the process is conducted, a more or less wide range of products is thereby produced, for example not only ethyne and hydrogen but also fractions of ethane, propane, ethene and other lower hydrocarbons. This allows the heat produced to be passed on for further use, such as the endothermic cracking of the hydrocarbons, to a much greater extent.

After this lowering of the temperature, for example to 150 to 300° C., solid constituents, in particular carbon particles, are separated and the gas mixture, which may, depending on the starting materials, contain not only ethyne and hydrogen but also further substances, such as ethene, ethane, carbon monoxide and volatile sulphur compounds, such as H₂S and CS₂, is passed on for further processing to obtain ethyne. Ethyne may in this case be separated from the gas mixture by selective absorption into a solvent. Suitable solvents are, for example, water, methanol, N-methyl pyrrolidone or mixtures thereof. Suitable methods for the separation of ethyne from the gas mixture are known from the prior art, for example from Ullmann's Encyclopaedia of Industrial Chemistry, Volume 1, 2012 Wiley-VCH Verlag GmbH & Co. KGaA, Weinheim, DOI: 10.1002/14356007.a01_{—}097.pub4, pages 291 to 293, 299 and 300, DE 31 50 340 A1 and WO 2007/096271 A1.

The power consumption of the electrothermic plant for the production of ethyne depends on the planned capacity for the production of acetylene. As in the case of most other chemical production technologies, the specific investment costs (costs for the investment in relation to the installed production capacity) fall with increasing size of the plant. Customary plant sizes for the production of acetylene lie in the range of a few 10,000 tonnes of acetylene to a few 100,000 tonnes of acetylene per year (based on full utilization). As the literature discloses, the specific energy requirement in the reaction part for the production of acetylene lies in the range from about 9 to about 12 MWh_{electrical} per tonne of ethyne, depending on the raw material used. Including the requirement for electrical energy for the processing, this gives the absolute power requirement of the acetylene plant. The desired production capacity is generally achieved by a parallel arrangement of multiple arc reactors, which may be controlled together or separately.

The integrated plant according to the invention also comprises a plant for electricity generation, to which a product gas that is obtained in the plant for the production of ethyne is fed via a conduit. All plants with which electrical power can be generated from the product gas are suitable here as plants for electricity generation. Preferably, a plant for electricity generation that has a high efficiency is used.

The product gas fed to the plant for electricity generation preferably contains hydrogen and/or hydrocarbons different from ethyne. The hydrocarbons may be unconverted feedstocks of the electrothermic production of ethyne, hydrocarbons added during quenching, hydrocarbons formed by the quenching or mixtures thereof.

In a preferred embodiment, the plant for electricity generation comprises a fuel cell. In this embodiment, a product gas that substantially consists of hydrogen is preferably fed to the plant for electricity generation.

In a further preferred embodiment, the plant for electricity generation comprises a power generating plant with a turbine. With particular preference, the plant comprises a gas turbine that can be operated with hydrogen and/or hydrocarbon-containing gases. Used with most preference is a gas turbine that can be operated with mixtures of hydrogen and hydrocarbon-containing gases of changing composition.

Preferably, the power generating plant with a turbine is a gas-and-steam turbine power plant, also known as a combined cycle gas-and-steam power plant. In these power generating plants, the principles of a gas turbine power plant and a steam power plant are combined. A gas turbine generally serves here inter alia as a heat source for a downstream waste heat boiler, which in turn acts as a steam generator for the steam turbine.

In addition to the product gas obtained in the production of ethyne, the plant for electricity generation may also be fed further substances, for example additional hydrogen for the operation of a fuel cell or additional fuel for the operation of a turbine or the heating of a steam generator.

The power output of the plant for electricity generation may be chosen depending on the production capacity of the plant for the electrothermic production of ethyne. Preferably, the output of the plant for electricity generation is chosen such that the power requirement of the plant for the electrothermic production of ethyne at full load is completely covered by the plant for electricity generation. In this case, the ratio of the electrical power output to the ethyne production capacity is preferably in a range from 2 to 20 MW_{electrical} per t/h of ethyne, with particular preference in a range from 5 to 15 MW_{electrical} per t/h of ethyne. The power can in this case be achieved by a single device or a combined group of multiple devices, where the combined group (pool) can be achieved by way of a common control system. Electrical energy for the plant for the electrothermic production of ethyne can also be drawn from the electricity network. Similarly, the plant for electricity generation may be dimensioned such that, in addition to the plant for the electrothermic production of ethyne, further electricity consumers are also supplied or the electrical energy surplus to the requirements of the plant for the electrothermic production of ethyne is fed into an electricity network.

In the integrated plant, the plant for the electrothermic production of ethyne is connected to the plant for electricity generation via a conduit, with which the plant for electricity generation is fed a product gas obtained in the plant for the electrothermic production of ethyne. The product gas preferably consists of hydrogen and/or hydrocarbon-containing gases. The product gas may be fed via the conduit to the plant for electricity generation in a gaseous or liquefied form, where the liquefaction may take place by increasing the pressure or reducing the temperature.

The conduit that connects the plant for the electrothermic production of ethyne to the plant for electricity generation preferably has a length of less than 10 km, with particular preference less than 1 km.

In a preferred embodiment, the plant for the electrothermic production of ethyne has a device for separating the gas mixture obtained in the electrothermic production, this device being connected to the plant for electricity generation. In the device for separating the gas mixture obtained in the electrothermic production of ethyne, ethyne is separated from hydrogen and other hydrocarbons. The mixture separated from ethyne and containing hydrogen and hydrocarbons may be fed directly to the plant for electricity generation. Alternatively, hydrogen may be separated from the mixture separated from ethyne and either hydrogen or a thereby resultant hydrocarbon-containing gas is fed to the plant for electricity generation. Similarly, hydrogen and a hydrocarbon-containing gas may also be fed via separate conduits from the device for separating the gas mixture obtained in the electrothermic production of ethyne to the plant for electricity generation. The separation of hydrogen and hydrocarbons may also take place incompletely in the integrated plant according to the invention, without incomplete separation having disadvantageous effects on the operation of the plant, so that the expenditure on apparatus and the energy consumption for the separation can be reduced in comparison with complete separation, such as that carried out in plants for the electrothermic production of ethyne according to the prior art.

In a preferred embodiment of the integrated plant, the plant for electricity generation comprises devices that are separate from one another for the generation of electricity from hydrogen and for the generation of electricity from a hydrocarbon-containing gas, which are preferably connected via separate conduits to a device for separating the gas mixture obtained in the electrothermic production of ethyne. With particular preference, the plant for electricity generation comprises a fuel cell for the generation of electricity from hydrogen and a gas-and-steam turbine power plant for the generation of electricity from a hydrocarbon-containing gas. In the case of this embodiment, gas-and-steam turbine power plants, which are not suitable for the conversion of hydrogen-rich gases into electricity, can also be used in the integrated plant according to the invention.

In a preferred embodiment, the integrated plant according to the invention additionally has at least one reservoir for a product gas obtained in the plant for the electrothermic production of ethyne, the reservoir being connected via conduits both to the plant for the electrothermic production of ethyne and to the plant for electricity generation. With particular preference, the reservoir is connected to the previously described device for separating the gas mixture obtained in the electrothermic production of ethyne, so that hydrogen and/or hydrocarbon-containing gases separated from ethyne can be stored in the reservoir. Preferably, the reservoir is a hydrogen reservoir. With particular preference, the integrated plant comprises both a hydrogen reservoir and a reservoir for hydrocarbon-containing gases separated from ethyne.

The integrated plant may additionally also comprise a device with which the composition of a product gas obtained in the electrothermic production of ethyne can be changed before it is fed to the plant for electricity generation. Preferably, the integrated plant additionally comprises a device with which hydrogen obtained as a co-product in the electrothermic production of ethyne can be converted into hydrocarbons by a Fischer-Tropsch synthesis or by methanation. The hydrocarbons obtained in this way may be fed to the plant for electricity generation together with hydrocarbons separated from ethyne or separately therefrom. A conversion of hydrogen into hydrocarbons simplifies the feeding of product gas obtained in the electrothermic production of ethyne in the case of plants for electricity generation in which hydrocarbons are burned for electricity generation and in which the content of hydrogen in the fuel gas must be kept within certain narrow limits for reliable operation. Suitable plants for Fischer-Tropsch synthesis or methanation are known from the prior art, for methanation for example from DE 43 32 789 A1 and WO 2010/115983 A1.

In a preferred embodiment, the integrated plant comprises in the plant for the electrothermic production of ethyne a steam generator, with which steam is generated from the waste heat of the electrothermic process, in the plant for electricity generation a device in which electricity is generated from steam, and a steam conduit, with which steam generated in the steam generator is fed to the device in which electricity is generated from steam. Preferably, an indirect quenching of the reaction gas obtained in an arc reactor is used as the steam generator. The device in which electricity is generated from steam is preferably a steam turbine or a steam motor and with particular preference a steam turbine. With most preference, the steam turbine is part of a gas-and-steam turbine power plant. With this embodiment, waste heat generated in the plant for producing ethyne can be used for generating electricity and the fuel requirement for operating the device in which electricity is generated from steam can be reduced.

In a preferred embodiment, the integrated plant according to the invention additionally comprises a reservoir for ethyne. This reservoir makes it possible to continue operating downstream reactions for converting ethyne into further products continuously, even when, at low electricity supply, only a little or no ethyne at all is produced in the plant for the electrothermic production of ethyne. The storage of ethyne preferably takes place by it being dissolved in a solvent, with particular preference in a solvent that is used for the absorption of ethyne in the separation of ethyne from the reaction mixture of the electrothermic production of ethyne.

In a further preferred embodiment, the integrated plant according to the invention is connected to a weather forecasting unit. Such a connection to a weather forecasting unit makes it possible to adapt the operation of the plant so as on the one hand to be able to make use of the possibility of using inexpensive surplus electricity and the possibility of providing electricity from the plant for electricity generation when there is a low electricity supply, and accordingly a high price for electricity, and on the other hand always to provide sufficient ethyne for the continuous operation of a downstream, ethyne-consuming plant. It is thus possible, depending on the result of the weather forecast, for example to bring a reservoir for ethyne to a high or low filling level. In addition, a plant for the further processing of the ethyne may be prepared and set up for modified operating modes. For instance, when there is a relatively long-term shortfall of electricity, these parts of the system can be set up for a reduced production capacity, so that an interruption in the operation owing to a lack of ethyne can be avoided.

In addition, the integrated plant may be connected to a unit for producing a consumption forecast, where this unit has with preference a data memory which comprises data on historical consumption. The data on historical consumption may comprise for example the daily variation, the weekly variation, the annual variation and further variations in terms of the electricity demand and/or the electricity generation. The data on the consumption forecast may also take into consideration specific changes, for example the gain or loss of a major consumer. In addition or as an alternative, the data memory may also contain data on the historical variation in electricity prices.

In the case of the method according to the invention for the flexible use of electricity, in the integrated plant according to the invention, at times of a high electricity supply, the plant for the electrothermic production of ethyne is operated and at least some of the hydrogen and/or gaseous hydrocarbons obtained in addition to ethyne is stored and, at times of a low electricity supply, stored hydrogen and/or gaseous hydrocarbons are fed to the plant for electricity generation. Preferably, the method involves storing hydrogen.

The electricity supply may take the form both of a surplus of electricity and a shortfall of electricity. A surplus of electricity is obtained if at a certain time more electricity from renewable energy sources is provided than the total consumption of electricity at this time. A surplus of electricity is also obtained if large amounts of electrical energy from fluctuating renewable energy sources are provided and the cutting back or shutting down of power generating plants involves high costs. An electricity shortfall is obtained if comparatively small amounts from renewable energy sources are available and inefficient power generating plants or power generating plants involving high costs have to be operated. The cases of a surplus of electricity and shortfall of electricity described here may become evident in various ways. For example, the prices on the electricity exchanges may be an indicator of the respective situation, a surplus of electricity leading to lower electricity prices and a shortfall of electricity leading to higher electricity prices. A surplus of electricity or shortfall of electricity may, however, also exist without there being any direct effect on the electricity price. For example, a surplus of electricity may also exist if the operator of a wind farm produces more power than it has predicted and sold. By analogy, there may be a shortfall of electricity if the operator produces less power than it has predicted. According to the invention, the terms surplus of electricity and shortfall of electricity cover all of these cases.

The method according to the invention is preferably operated such that at least some of the electricity required for the electrothermic production of ethyne is generated by the plant for electricity generation comprised by the integrated plant from product gas that is obtained in the electrothermic production of ethyne. If the plant for the electrothermic production of ethyne is operated at times of a high electricity supply, the plant for electricity generation comprised by the integrated plant is preferably operated with reduced output or shut down, and a greater part of the electricity required for the electrothermic production of ethyne is taken from an electricity network with a high electricity supply. By analogy, if the plant for electricity generation comprised by the integrated plant is operated at times of a low electricity supply, the plant for the electrothermic production of ethyne is preferably operated with reduced output or shut down, and a smaller part of the electricity required for the electrothermic production of ethyne is taken from the electricity network or electricity from the plant for electricity generation comprised by the integrated plant is fed into the electricity network.

The storing of hydrogen and/or gaseous hydrocarbons obtained in addition to ethyne preferably takes place in a reservoir comprised by the integrated plant, with particular preference in a reservoir arranged between the plant for the electrothermic production of ethyne and the plant for electricity generation as described above. Alternatively, the storage may, however, also take place in a separate reservoir that is connected to the integrated plant via a gas distributing conduit, for example a natural gas network.

The type of reservoir is not critical, and so a pressurized tank, a liquefied gas reservoir, a reservoir in which hydrocarbons are absorbed in a solvent or a reservoir with gas adsorption on a solid may be used for this. Also suitable for the storage of hydrogen are chemical reservoirs, in which hydrogen is stored by a reversible chemical reaction. Preferably, separate reservoirs are used for hydrogen and for gaseous hydrocarbons obtained in addition to ethyne. The capacity of the reservoir is preferably dimensioned to hold the amount of hydrogen and/or gaseous hydrocarbons produced by the plant for the electrothermic production of ethyne at full load within 2 hours, with particular preference the amount produced within 12 hours and with most particular preference the amount produced within 48 hours.

In a preferred embodiment of the method according to the invention, the plant for the electrothermic production of ethyne has an arc reactor and the gas mixture obtained from the arc reactor is mixed with a hydrocarbon-containing gas and/or a hydrocarbon-containing liquid for cooling. In this case, as described above, at least some of the hydrocarbons are cracked endothermically, thus obtaining cracking products that have a higher energy content than the starting materials and deliver a greater amount of electrical energy if fed to the plant for electricity generation than if the starting materials were fed to it. This embodiment thus makes it possible to store electrical energy fed to the arc reactor in the form of high-energy cracking products. Preferably, the type and/or amount of hydrocarbon-containing gas and/or liquid is chosen in dependence on the expected electricity supply. This is particularly advantageous in the case of a method in which direct quenching by mixing with hydrocarbon-containing gas and/or liquid is used in combination with indirect quenching with steam generation, since it is then possible to control which fraction of the heat stemming from the arc reactor is stored in the form of cracking products for later electricity generation and which fraction is used in the form of steam for immediate electricity generation without storage by choosing the type and/or amount of the hydrocarbons added in the direct quenching.

Preferably, when there is a high electricity supply, the electrical energy used for the production of ethyne originates at least partially from renewable energy sources, with particular preference from wind power and/or solar energy. However, it should be noted that, according to current German legislation, electricity that has been obtained from renewable energy sources may be fed into the electricity network even without any demand at the particular time and must be paid for. Therefore, conventionally generated electricity may at times constitute a “surplus”, since it may be less profitable for a power plant operator to run a power plant down to a low output than to sell electricity below the cost price. This surplus electrical energy obtained from the continued operation of conventional plants can be economically used by the present method, in particular stored.

In a preferred embodiment of the method according to the invention, a gas-and-steam turbine power plant is used as the plant for electricity generation and, when there is a high electricity supply, the plant for the electrothermic production of ethyne is operated with an output of over 80% of the rated capacity and the plant for electricity generation is operated at 0-50% of the rated electrical capacity and, when there is a low electricity supply, the plant for the electrothermic production of ethyne is operated with an output of 0-50% of the rated capacity and the plant for electricity generation is operated at over 80% of the rated electrical capacity.

When there is a high electricity supply, the gas-and-steam turbine power plant is operated preferably with an output of at most 40% and with particular preference at most 30% of the rated electrical capacity.

When there is a low electricity supply, the plant for the electrothermic production of ethyne is operated preferably with an output of at most 40% and with particular preference at most 30% of the rated capacity.

If the gas-and-steam turbine power plant is operated with combined heat and power generation, the rated electrical capacity of the power plant may be set either by changing the amount of gas used or by changing the proportion of steam taken as process steam and not used for electricity generation.

Expediently, in the greatest part of the operating time, when there is a moderate electricity supply, both the plant for the electrothermic production of ethyne and the plant for electricity generation are operated with an output at which the total amount of hydrogen and/or gaseous hydrocarbons obtained in addition to ethyne in the plant for the electrothermic production of ethyne is fed to the plant for electricity generation.

This design of the method according to the invention allows a high operating time both of the plant for the electrothermic production of ethyne and of the plant for electricity generation, and consequently economical operation of both plants, to be achieved.

The method according to the invention preferably comprises the steps of
a) setting a first threshold value and a second threshold value for an electricity supply,
b) determining the electricity supply,
c) changing the electrical power output of the plant for electricity generation in dependence on the electricity supply if the electricity supply exceeds the first threshold value and changing the output of the plant for the electrothermic production of ethyne in dependence on the electricity supply if the electricity supply is below the second threshold value, and
d) repeating steps b) and c).

The threshold values are preferably set depending on the filling level of the reservoir for ethyne at the particular time or depending on the predictions for the development of the consumption and generation of ethyne in the next hours. If, for example, the filling level of the reservoir for ethyne falls to a low value, the threshold value below which the output of the plant for the electrothermic production of ethyne is reduced is set to a lower value.

The electricity supply may be determined either directly by agreement with electricity generators and/or electricity consumers or indirectly by way of trading platforms and/or by OTC methods and an associated electricity price. In a preferred embodiment, the electricity supply is determined by agreement with generators of electricity from wind energy and/or solar energy. In a further preferred embodiment, the electricity supply is determined by way of the electricity price on a trading platform.

If the electricity supply is determined by agreement with generators of electricity from wind energy and/or solar energy, preferably the electrical power output of the plant for electricity generation is changed in accordance with the surplus of electricity when the first threshold value is exceeded and the output of the plant for the electrothermic production of ethyne is changed in accordance with the shortfall of electricity when the second threshold value is not reached.

If the electricity supply is determined by way of the electricity price on a trading platform, preferably the electrical power output of the plant for electricity generation is changed to a predetermined lower value when the first threshold value is exceeded and the output of the plant for the electrothermic production of ethyne is changed to a predetermined lower value when the second threshold value is not reached.

The absolute level of the first threshold value from which a reduction of the output of the plant for electricity generation takes place is not important for this embodiment of the present method and can be set on the basis of economic criteria. The same applies to the second predetermined value, below which a reduction of the output of the plant for the electrothermic production of ethyne takes place.

If the output of the two plants is coordinated with each other, the first predetermined threshold value and the second threshold value are preferably chosen to be the same.

The electricity supply is preferably calculated in advance from the data of a weather forecast. On the basis of the electricity supply calculated in advance, the aforementioned threshold values for an electricity supply are then preferably chosen such that, in the time period of the forecast, on the one hand a planned amount of ethyne is produced and on the other hand the storage capacity for hydrogen and/or gaseous hydrocarbons obtained in addition to ethyne is not exceeded.

Joint operation of the plant for the electrothermic production of ethyne and the plant for electricity generation when there is a moderate electricity supply surprisingly allows high operating times to be attained, so that a high level of profitability of the plant is achieved.

Within a calendar year, the plant for electricity generation is operated preferably for at least 4000 full-load hours, with preference at least 5000 full-load hours and with particular preference at least 5500 full-load hours. The full-load hours are in this case calculated according to the formula

full-load hours=W/P

where W is the electrical work in MWh provided within a calendar year and P is the rated electrical capacity of the plant in MW.

If the plant for the electrothermic production of ethyne comprises at least one arc reactor, within a calendar year the arc reactors are operated preferably on average for at least 2500 full-load hours, with preference at least 4000 full-load hours and with particular preference at least 5000 full-load hours. The full-load hours are in this case calculated according to the formula

full-load hours=production/capacity

where “production” denotes the amount of ethyne in tonnes produced within a calendar year and “capacity” denotes the total rated capacity of the arc reactors in tonnes of ethyne per hour.

Further preferred embodiments of the method according to the invention arise from the description given above of an integrated plant according to the present invention.

The present integrated plant and the method are suitable for the production of ethyne in a very economical and resource-conserving way. Ethyne can be transformed into many valuable intermediate products, while it is possible in this way to achieve a surprising reduction in the carbon dioxide emissions.

This surprising reduction is based on a number of synergistically acting factors. These include the fact that electricity from renewable energy sources can be used for the production of ethyne, allowing the production of ethyne to be adapted very flexibly to an electricity supply. Furthermore, hydrogen can be obtained with a very high electricity efficiency, and can be used for generating electrical energy without the release of carbon dioxide. Furthermore, heat is often released in the production of the valuable derivatives. This waste heat can often be used to cover the heat requirement in other parts of the process (for example in the case of distillative separation processes). The emission of carbon dioxide is reduced correspondingly if, on the other hand, an oxidation of hydrocarbons were necessary to generate the process heat. The specific enthalpy is higher in the case of ethyne than in the case of other conventional hydrocarbons that are alternatively used for the synthesis of the same end products, such as for example ethylene or propylene. Consequently, more waste heat can generally be generated in the conversion and used for other applications.

Furthermore, it may be provided that the ethyne generated is used for the production of acetone, butanediol or unsaturated compounds with a molecular weight of at least 30 g/mole. The unsaturated compounds with a molecular weight of at least 30 g/mole particularly include vinyl ethers, preferably methyl vinyl ether or ethyl vinyl ether; vinyl halides, preferably vinyl chloride; acrylonitrile; unsaturated alcohols, preferably allyl alcohol, propargyl alcohol, butynediol and/or butenediol; vinyl acetylene, acrylic acid and acrylic acid ester; esters of vinyl alcohol, preferably vinyl acetate; butadiene and butene.

The ethyne generated may also be hydrogenated selectively to ethene.

Furthermore, byproducts from these processes can be used for the generation of electricity. Gaseous byproducts or suitable liquid byproducts after vaporization may preferably be fed here into the gas turbine. Solid residues may be converted into combustible gases, in particular using hydrogen, and subsequently converted into electricity in a gas turbine. Preferably, the ethyne produced in the plant for the electrothermic production of ethyne is converted in at least one further process into a further product and a byproduct from this process is used in the plant for electricity generation for the generation of electricity.

Furthermore, the waste heat obtained in a reaction of the ethyne to form an unsaturated compound with a molecular weight of at least 30 g/mole or another derivative may be used at least partially for the generation of electricity. Preferably, the ethyne generated in the plant for the electrothermic production of ethyne is converted in at least one further process into a further product and heat generated during this process is used in the plant for electricity generation for the generation of electricity.

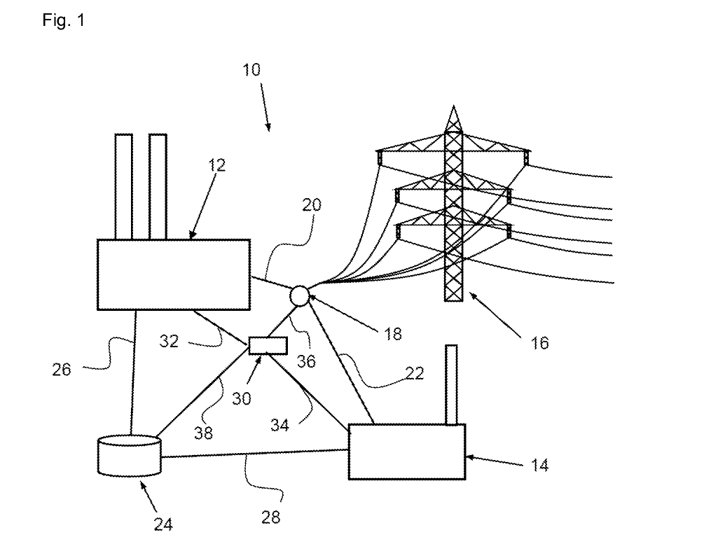

Preferred embodiments of the present invention are explained by way of example below on the basis of FIGS. 1.

FIG. 1 shows a schematic structure of an integrated plant according to the invention.

FIG. 1 shows a schematic structure of an integrated plant 10 according to the invention, comprising a plant for the electrothermic production of ethyne and a plant 14 for electricity generation, the integrated plant 10 being connected to a central electricity network 16. The individual devices may be connected here directly to the central electricity network 16 or, as shown in FIG. 1, be connected to the central electricity network 16 via a switching point 18 for electricity transmission. The plant 12 for the electrothermic production of ethyne is then connected via a first electrical connecting line 20 to the switching point 18 for electricity transmission, the plant 14 for electricity generation is connected via a second electrical connecting line 22 to the switching point 18 for electricity transmission and the switching point 18 for electricity transmission is connected to the central electricity network 16. This embodiment may have advantages in the installation costs and/or the operating expenditure.

In the embodiment shown in FIG. 1, the integrated plant 10 comprises a hydrogen reservoir 24, which may be filled with hydrogen from the plant 12 for the electrothermic production of ethyne via a first connecting conduit 26 for hydrogen. For generating electrical energy, the hydrogen stored in the hydrogen reservoir 24 may be fed to the plant 14 for electricity generation via the second connecting conduit 28 for hydrogen.

Furthermore, in the embodiment shown, the integrated plant 10 has a control system 30, which is connected via a first communication connection 32 to the plant 12 for the electrothermic production of ethyne, via a second communication connection 34 to the plant 14 for electricity generation, via a third communication connection 36 to the switching point 18 for electricity transmission and via a fourth communication connection 38 to the hydrogen reservoir 24.

The features of the invention that are disclosed in the description above and the claims, figures and exemplary embodiments can also be used in any desired combination for carrying out the invention.

### LIST OF REFERENCE SIGNS

10 integrated plant
12 plant for the electrothermic production of ethyne
14 plant for electricity generation
16 central electricity network
18 switching point for electricity transmission
20 first electrical connecting line
22 second electrical connecting line
24 hydrogen reservoir
26 first connecting conduit for hydrogen
28 second connecting conduit for hydrogen
30 control system
32 first communication connection
34 second communication connection
36 third communication connection
38 fourth communication connection

1.-21. (canceled)

22. An integrated plant, comprising a plant for the electrothermic production of ethyne and a plant for electricity generation, characterized in that said plant for the electrothermic production of ethyne is connected to said plant for electricity generation via a line and the line feeds a product gas obtained in said plant for the electrothermic production of ethyne to said plant for electricity generation.

23. The integrated plant of claim 22, wherein said plant for electricity generation comprises a fuel cell.

24. The integrated plant of claim 22, wherein said plant for electricity generation comprises a power generating plant with a turbine.

25. The integrated plant of claim 24, wherein said power generating plant with a turbine comprises a gas turbine that can be operated with any of hydrogen, hydrocarbon-containing gases or mixtures thereof.

26. The integrated plant of claim 24, wherein said power generating plant with a turbine is a gas-and-steam turbine power plant.

27. The integrated plant of claim 22, wherein said plant for the electrothermic production of ethyne comprises an arc reactor.

28. The integrated plant of claim 22, wherein said plant for the electrothermic production of ethyne comprises a device for separating the gas mixture obtained in the electrothermic production and said device for separating the gas mixture obtained in the electrothermic production is connected to said plant for electricity generation.

29. The integrated plant of claim 22, comprising at least one store for any of hydrogen, hydrocarbon-containing gases or mixtures thereof that were separated from ethyne, which store is connected via a line to said plant for the electrothermic production of ethyne and via a line to said plant for electricity generation.

30. The integrated plant of claim 22, wherein said plant for the electrothermic production of ethyne comprises a steam generator, with which steam is generated from waste heat of the electrothermic process, said plant for electricity generation comprises a device in which electricity is generated from steam, and the integrated plant comprises a steam line, with which steam generated in said steam generator is fed to said device in which electricity is generated from steam.

31. The integrated plant of claim 22, wherein said integrated plant is connected to a weather forecasting unit.

32. A method for the flexible use of electricity, wherein in an integrated plant as claimed in claim 22, at times of a high electricity supply, the plant for the electrothermic production of ethyne is operated and at least some of the hydrogen, the gaseous hydrocarbons obtained in addition to ethyne or both are stored and, at times of a low electricity supply, stored hydrogen, gaseous hydrocarbons ore both are fed to the plant for electricity generation.

33. The method of claim 32, wherein said plant for the electrothermic production of ethyne has an arc reactor, and the gas mixture obtained from the arc reactor is mixed with a hydrocarbon-containing gas or a hydrocarbon-containing liquid for cooling.

34. The method of claim 33, wherein the composition, the amount or both of the gas or liquid is chosen in dependence on the expected electricity supply.

35. The method of claim 32, wherein the electricity supply is calculated in advance from the data of a weather forecast.

36. The method of claim 32, wherein said plant for electricity generation is a gas-and-steam turbine power plant and, in the integrated plant, when there is a high electricity supply, said plant for the electrothermic production of ethyne is operated with an output of over 80% of the rated capacity and said plant for electricity generation is operated at 0-50% of the rated electrical capacity and, when there is a low electricity supply, said plant for the electrothermic production of ethyne is operated with an output of 0-50% of the rated capacity and said plant for electricity generation is operated at over 80% of the rated electrical capacity.

37. The method of claim 32, comprising the steps of
a) fixing a first threshold value for an electricity supply and a second threshold value for an electricity supply,
b) determining the electricity supply,
c) changing the electrical power output of said plant for electricity generation in dependence on the electricity supply if the electricity supply exceeds said first threshold value and changing the output of said plant for the electrothermic production of ethyne in dependence on the electricity supply if the electricity supply is below said second threshold value, and
d) repeating steps b) and c).

38. The method of claim 37, wherein the first threshold value and the second threshold value are the same.

39. The method of claim 32, wherein said plant for the electrothermic production of ethyne comprises at least one arc reactor and, within a calendar year, the arc reactors are operated on average for at least 2500 full-load hours.

40. The method of claim 32, wherein, within a calendar year, said plant for electricity generation is operated for at least 4000 full-load hours.

41. The method of claim 32, wherein the ethyne produced in said plant for the electrothermic production of ethyne is converted in at least one further process into a further product and a byproduct from this process is used in said plant for electricity generation for the generation of electricity.

42. The method of claim 32, wherein the ethyne produced in said plant for the electrothermic production of ethyne is converted in at least one further process into a further product and heat generated during this process is used in said plant for electricity generation for the generation of electricity.
